(19) United States
(12) Patent Application Publication
(10) Pub. No.: US 2007/0162150 A1
(43) Pub. Date: Jul. 12, 2007

(54) MODULAR PROSTHETIC IMPLANT FOR UPPER AND LOWER EXTREMITY AMPUTEES

(76) Inventors: John Richard Fago, Bethel, VT (US); Julie Patterson Fago, Bethel, VT (US); Richard M. Greenwald, Norwich, VT (US); Michael B. Mayor, Lebanon, NH (US)

Correspondence Address: BARLOW, JOSEPHS & HOLMES, LTD., 101 DYER STREET, 5TH FLOOR, PROVIDENCE, RI, 02903, US

(21) Appl. No.: 11/622,273

(22) Filed: Jan. 11, 2007

Related U.S. Application Data
(60) Provisional application No. 60/758,448, filed on Jan. 12, 2006.

(51) Int. Cl. A61F 2/30; A61F 2/74

(52) U.S. Cl. 623/23.42; 623/27

(57) ABSTRACT

The invention is generally directed to the novel and unique modular implant that provides an improved surface for interfacing with an external prosthesis. The implant includes a stem, an optional extension member connected to the stem, a prosthetic condyle, or in the case of application to other levels of cut bone amputations, a location-specific terminal shape, connected to the extension member. The stem is inserted directly into the canal of the bone and tissue, including muscle, is attached directly to the prosthetic and the stem is inserted directly into the canal of the bone. The simulated anatomically correct condyle better distributes the load of the patient's weight and avoids twisting thereby relieving pain to improve the functional connection between the residual limb and an external prosthesis in all levels of upper and lower extremity amputations. The surgically implanted internal prosthetic simplifies and lowers the cost of rehabilitation and results in an improvement in the function of all existing external prosthetic components and systems.

### CROSS REFERENCE TO RELATED APPLICATION

This application claims the benefit of the Provisional Application No. 60/758,448, filed Jan. 12, 2006.

### BACKGROUND OF THE INVENTION

The present invention relates to a surgically implanted prosthetic device for the distal end of the cut bone for amputees that can be implanted to improve functional rehabilitation for all levels of lower and upper extremity amputation. More particularly, it relates to an implant designed to be application specific that provides increased surface area and more anatomic geometry for external loading, that resists rotation of an externally fit prosthesis, and a method for soft tissue fixation to improve amputee function.

There are approximately 180,000 lower limb amputations performed annually, of which approximately 45,000 are above the knee, also known as “AK” as reported by the Amputee Coalition of America—National Limb Loss Information Center in 2002 at http://www.amputeecoalition.org/fact sheets/limbloss us.html. A common and significant problem among lower limb amputees, and above knee amputees in particular, is a lack of a consistent and stable prosthesis fit. This problem has been well documented by Geil, MD in Consistency and accuracy of measurement of lower-limb amputee anthropometrics, J Rehabil Res Dev. 2005 March/April; 42(2): 131-140. A poorly fitting prosthesis can lead to discomfort, difficulty in ambulation, pain, localized areas of high pressure, and skin ulceration. This can lead to a decrease in activity level for the amputee, beginning a debilitating cycle compromising the long-term health of that individual. Disabled persons whose activities are limited complain of increased pain, depression, anxiety, insomnia, and grief. Above-knee prosthetic sockets depend upon critical fit with small tolerance for limb volume change. Similar problems exist for below-knee amputees for the same reasons.

As a result, users frequently suffer chronic recurrent soft tissue complications from unnatural dynamic loading. For lower-limb amputees, this recurrent soft tissue trauma occurs in sensitive areas essential for mid-stride weight bearing and results from unnatural loading of the soft tissue in its role assisting in the transfer of the patient's body weight down into the prosthetic leg. This stressful compression of soft tissue alternates with an also stressful stretching of the soft tissue during the other half of the walking cycle when the soft tissue supports the suspension of the prosthetic leg while the artificial leg is in the swing phase. This cycle produces an unwanted vertical “pistoning” of the residual limb within the prosthetic socket that leads inevitably to skin breakdown, skin appendage infection, user discomfort and a limitation of function and prosthesis use.

As a result, of the foregoing issues, a primary factor in the rehabilitation of an amputee, particularly a lower limb amputee, is the fit of the prosthesis. This issue is documented by Klute G, Kallfelz C, Czerniecki J. in Mechanical Properties of Prosthetic Limbs: Adapting to the patient. J Rehabil Res Dev. 2001 May-June; 38(3): 299-307. While certain improvements in socket design and material construction have enhanced amputee comfort, satisfaction, and security, poor fit remains one of the most vexing problems for lower limb amputees as stated by Legro M W, Reiber G, del Aguila M, Ajax M J, Boone D A, Larsen J A, Smith D G, Sangeorzan B. in Issues of Importance reported by persons with lower limb amputations and prostheses. J Rehabil Res Dev. 1999; 36(6): 155-63. Also, socket discomfort is reported to be the most common complaint in the prosthetist's office which is addressed by Hanspal R, Fisher K, Nieveen R. in Socket Fit Comfort Score. Paper presented at the IX World Congress ISPO, Amsterdam, Netherlands 1998. In fact, 74% of all lower-limb amputees report residual limb pain, with 38% percent describing the pain as severe as noted by Ehde D M, Czerniecki J M, Smith D G, Campbell K M, Edwards W T, Jensen M P, Robinson L R. in Chronic Phantom sensations, phantom pain, residual limb pain, and other regional pain after lower limb amputation. Arch Phys Med Rehabil. 2000 August; 81. Secondary complications arising from poor fit include blisters, cysts, furnacles, and dermatitis, all of which occur at a very high rate in the AK amputee population.

More specifically, most above knee amputations involve cutting the femur 14 transversely at the required level of amputation, as seen in FIGS. 1 and 2. Subsequently, myodesis/myoplasty is typically performed by suturing the agonists and antagonists of the knee at 28 together over the distal end 20a of the cut femur. Other muscles 22 may also be sutured to the distal bundle but are often left unattached. The residual limb of the prior art, generally referred to as 17, remains.

Shortly after amputation, the prior art residual limb 17 is typically fit with a temporary prosthesis (not shown) to control edema. During the early postoperative period, the residual limb 17 undergoes rapid remodeling and changes in size and shape. The use of temporary prostheses and initial weightbearing in a socket is encouraged and helps to reduce post-operative edema, to gain the advantages of rapid restoration of ambulation and body image, and because maturation of the residual limb is enhanced by the support accorded it. An external prostheses 12 for accommodating the limb 17 of FIGS. 1 and 2 is shown generally in FIG. 8. This external prosthesis 12 can be modified to accommodate and type of residual limb.

The relationship between function, comfort and tissue health of the lower limb residuum relates to the biomechanical interface between the skin and the prosthesis. The lower-limb prosthetic socket, such as that shown in FIG. 8, is effectively a fixed volume into which the residual limb is placed. Normal fluctuations in residual limb volume result in a socket that is either too tight or too loose, respectively, which is a significant problem in prior art prosthesis attempts. The ability to bear weight on the distal end of the residual limb represents a major challenge to the amputee and creates difficulties for creating an effective and comfortable prosthesis.

Typically, an AK amputee has minimal ability to directly bear weight on the distal end of the residuum, usually less than 5% of body weight. The prosthetist attempts to create a well fitting socket that provides load distribution across a roughly horizontal line between the ischium and the trochanter, and in a properly adducted socket, along the lateral side of the femur, to make up for the inability to directly bear weight distally. Unfortunately, this leads to unnatural loading of soft tissue and often discomfort, particularly in the area of the groin, thigh, and buttocks. Additionally, the suspension of the residual limb in the socket and the load distribution that occurs during ambulation are affected by the shape of distal end of the residuum and the overlying soft tissue.

Another critical variable in the ability to bear weight on the residual limb for a lower limb amputee, and particularly in the success of knee disarticulations, also known as KD, is the availability of quality soft tissue at the distal end to support weightbearing. Thus, there is a desire for a modular approach that allows different levels of amputation and subsequent implant length allowing the surgeon to effectively utilize the available soft tissue at the distal end of the amputation to ensure functional success for the resultant weight-bearing distal prosthetic condyle. Prior art devices and methods are not capable of meeting these needs.

Data suggests the importance of direct distal weight bearing. Trans-tibial amputees achieve 87% of normal self selected walking speed as compared to 63% for transfemoral amputees suggesting that the higher the level of amputation the slower the self selected walking speed. This is supposed by Walters, et al. in the Journal of Bone and Joint Surgery, 1976. However, in certain functional realms, KD amputees out-perform trans-tibial amputees who retain the complete knee but reply upon proximal weight transfer at the tibial condyle flare. Specifically, study of forefoot impulse as the expression of walking propulsion suggests that below the knee, also known as BK, amputees achieve 93% of body weight while KD amputees averaged 98% body weight at prosthetic mid-stride. Even more significant was a finding that linear acceleration of the center of body weight during propulsion in BKs is 73% while KDs averaged 96%. This issue is addressed Orthodpedics, 1993 August:16(8):875-9. In recent years, the development of prosthetic components such as computer controlled prosthetic knees and energy storing/releasing lower legs/feet have led to marked improvement of function for AK amputees. However, these prior art attempts are still inadequate to fully address the problems associated with such prostheses.

Thus, the most significant problems in current prosthetic sockets involve weight transfer. First is the common problem of soft tissue bearing unnatural loads under stress that leads inevitably to tissue breakdown. Second, and perhaps more important in terms of being an opportunity for marked mechanical and functional improvement for the amputee, is the inability to have distal skeletal weight transfer in a direct gravitational line downward through the artificial leg to the ground.

Therefore, there can be significant benefits from hardware advances that make weight transfer from residual limb to prosthetic leg the place of greatest opportunity to achieve the next step in significant improvement for rehabilitative outcome in AK amputees.

In view of the foregoing, current surgical techniques for amputation of the distal femur above the knee do not provide an optimum solution for interfacing with a prosthetic socket for weight bearing and ambulation. In order to improve distal weightbearing, reduce rotation of the socket around the residuum and improve clinical function for above-knee amputees.

Therefore, there is a need for an implant for a prosthesis that addresses all of the problems associated with the prior art.

Therefore, there is a need for a implant that can be placed into the distal end of an amputated limb, such as a femur, for interfacing with a prosthesis.

There is also a need for a prosthetic implant that provides a better interface to the distal end of the limb for fitting to a prosthesis.

There is another need for a prosthetic implant that aids in avoiding rotational or twisting of the limb relative to an external prosthesis.

There is yet another need for a prosthetic implant that simulates more closely the anatomy of distal femur geometry.

A further need is for a prosthetic implant that mimics the result of a knee disarticulation.

Yet another need is for a prosthetic implant that is more conducive to muscle attachment and muscle function postoperatively.

There is a need for an improved interface surface to enable better weight distribution and reduce pain.

There is also a need for a method for providing such a prosthetic implant.

### SUMMARY OF THE INVENTION

The present invention preserves the advantages of prior art prosthetic implant systems. In addition, it provides new advantages not found in currently available prosthesis and implant systems for amputees and overcomes many disadvantages of such currently available systems.

The invention is generally directed to the novel and unique implantable condyle that is designed to provide an improved surface for interfacing with a prosthesis and for the replacement of a structure and structural function that is lost at the time of amputation. While the present invention is specifically developed for the lower extremity, it is also directly applicable for upper extremity amputees with cut bone or disarticulation amputations, where overlying soft tissue injury, rotation of an external prosthesis, and improved connection between the residual limb and the external prosthesis are desired and improved where rotation monitoring of other body parts, or the body in general. Use for upper extremity amputations is envisioned and considered within the scope of the present invention.

Upper extremity amputees share many of the same functional obstacles that result from a lack of a terminal cut bone structure to transfer force from the residual limb to the external prosthesis, as well as length issues that are the result of disarticulation. Principal among these are functional limitations imposed by surgical necessities that require a short residual cut bone length, a lack of strength contribution from not otherwise utilized muscles, and a lack of structural contribution from the cut bone to control the movement and function of the prosthesis. The utilization of the present invention for upper extremity amputees provides improved connection and control by the residual limb of the external prosthesis.

The system of the present invention is designed to improve the connection between a residual limb and an external prosthesis in all levels of upper and lower extremity amputations. This enhancement is novel in that the improvements are the result of a surgically implanted internal prosthetic that has the consequence of simplifying and lowering the cost of all subsequent external prosthetic rehabilitation and results in an improvement in the function of all existing external prosthetic components and systems.

Also, the system of the present invention is designed to fundamentally change the mechanism of weight and force transfer from the residual limb to the external prosthesis. By providing a location specific designed internal prosthetic structure for the end of an amputated cut bone, it allows gravitational body weight and muscular forces to more naturally interface with the external prosthesis. Improvements in function and comfort come from being able to return to more natural pathways of energy and force transmission between the residual limb and the prosthesis which are also now enhanced by greater contributions from not otherwise utilized muscles.

The present invention overcomes problems associated with existing interfaces that employ merely a cut bony end with muscles tied thereacross. The system of the present invention is designed to reduce the incidence of soft tissue breakdown between the residual limb and an external prosthesis. It is novel in that it produces this advantage by providing an internal structure that utilizes existing technologies and accepted surgical practice and requires no maintenance or replacement while permanently changing for the better the mechanism of weight and force transfer from the residual limb to the external prosthesis.

In accordance with the present invention, a new surgical method and device, including a modular implant, provides a more anatomical distal femur geometry. Internal surgical fixation is achieved and the result of knee disarticulation amputation is mimicked to gain advantages of this procedure over traditional transfemoral amputation methods that transversely cut the femur.

Unlike BK (below-the-knee) amputees who are able to bear much of their weight directly on the bony flare of the femoral condyles, AK (above-the-knee) amputees lack an available and usable bone structure to allow any direct weight transfer from the residual limb to the prosthetic leg. A surgical implanted prosthetic condyle, in accordance with the present invention, at the point of cut bone will enable transfemoral AK amputees (who make up the majority of above knee amputations) to gain the possibility of direct distal weight bearing. In addition, a longer more powerful skeletal/prosthetic lever with attachment of not otherwise utilized muscles leading to dramatic functional improvements previously only available to the small number of AK amputees at the level of KD (knee disarticulation). Unlike current KD amputations that result in uneven prosthetic/sound-side knee centers, the current invention allows even prosthetic and sound side knee centers thereby eliminating the major drawback for KD amputees who otherwise enjoy superior efficiency, endurance, comfort and reliability as compared to transfemoral AK amputees. The present invention enables such sound knee centers as outlined by Cull, D L, Taylor S M, Hamontree, S E, Langan E M, Snyder B A, Sullivan, T M, Youkey J R in A reappraisal of a modified through-knee amputation in patients with peripheral vascular disease; The American Journal of Surgery. 2001:182, 44-48. Bowker, J M, San Giovanni T P, Pinzur, M S.; North American Experience with Knee Disarticulation with Use of a Posterior Myofasciocutaneous Flap; and Healing Rate and Functional Results in Seventy-Seven Patients. The Journal of Bone and Joint Surgery, Incorporated 2000: 82-A(11).

In accordance with the present invention, a new and novel modular prosthetic implant and system is provided. The implant includes a stem, an optional extension which can be sized as desired and a condyle for receipt of muscle thereto. It is also possible that the extension is not needed at all depending on the length of the existing limb and what is needed for the implant. It is also possible that the stem can be integrally formed with the condyle portion to provide a unitary structure. The condyle at the free end is preferably made of a porous mesh material, such as that made of titanium or tantalum, to facilitate soft tissue ingrowth for permanent muscle attachment. As will be discussed in detail below, the implant system is modular in nature and the stem is implanted directly into the canal of the distal end of the femur. Muscle is surgically attached to the condyle for natural ongrowth or ingrowth thereon and for preparation as a surface for indirectly interfacing with the external prosthesis through the soft tissue.

It is therefore an object of the present invention to provide an implant for a prosthesis that addresses all of the problems associated with the prior art.

Therefore, an object of the present invention is to provide a implant that can be placed into the distal end of an amputated limb, such as a femur, for interfacing with an external prosthesis.

Another object of the present invention is to provide an implant that provides a better interface to the distal end of the limb for fitting to an external prosthesis.

A further object of the present invention is to provide an implant that aids in avoiding rotational or twisting of the limb relative to an external prosthesis.

Another object of the present invention is to provide a implant for a prosthesis that simulates more closely the anatomy of distal femur geometry and mimics the result of a knee disarticulation.

In applications at other levels of amputation, such as but not limited to, below the knee or below the elbow, another object of the present invention is to produce a prosthetic implant component that provides a location specific terminal shape to maximize efficient weigh and force transfer, comfort and user control of the external prosthesis.

An object of the present invention to provide an improved system for use with an implant for interfacing with a prosthesis that is more conducive to muscle attachment and growth.

A further object of the present invention is to provide an implant with an improved and larger load bearing surface to enable better weight distribution and to reduce pain.

There is also a need for a method for providing such a prosthetic implant.

It is yet another object of the present invention to stabilize the myodesis/myoplasty closures by both providing additional surface area for configuring the soft tissues, and providing porous fixation for those soft tissues to stabilize them as active muscular forces return.

Another object of the present invention is to provide a muscle reattachment area on the prosthesis for surgical reattachment of cut muscle.

### BRIEF DESCRIPTION OF THE DRAWINGS

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a front perspective view of a leg that has been amputated above the knee in accordance with known surgical procedures in the prior art;

FIG. 2 is a bottom perspective view showing the prior art technique of bone shaping and muscle reattachment for a knee disarticulation;

FIG. 3 is a front view of a leg with the modular implant of the present invention installed using a short extension member;

FIG. 4 is a front view of a leg with the modular implant of the present invention installed using a long extension member;

FIG. 5 is a front view of a first embodiment of the implant in accordance with the present invention;

FIG. 6 is a front view of a second embodiment of the implant in accordance with the present invention;

FIG. 7 is a front view of a third embodiment of the implant in accordance with the present invention; and

FIG. 8 is a perspective view of an example of an external prosthetic with a socket that can receive a limb equipped with the implant of the present invention.

### DETAILED DESCRIPTION OF THE PREFERRED EMBODIMENT

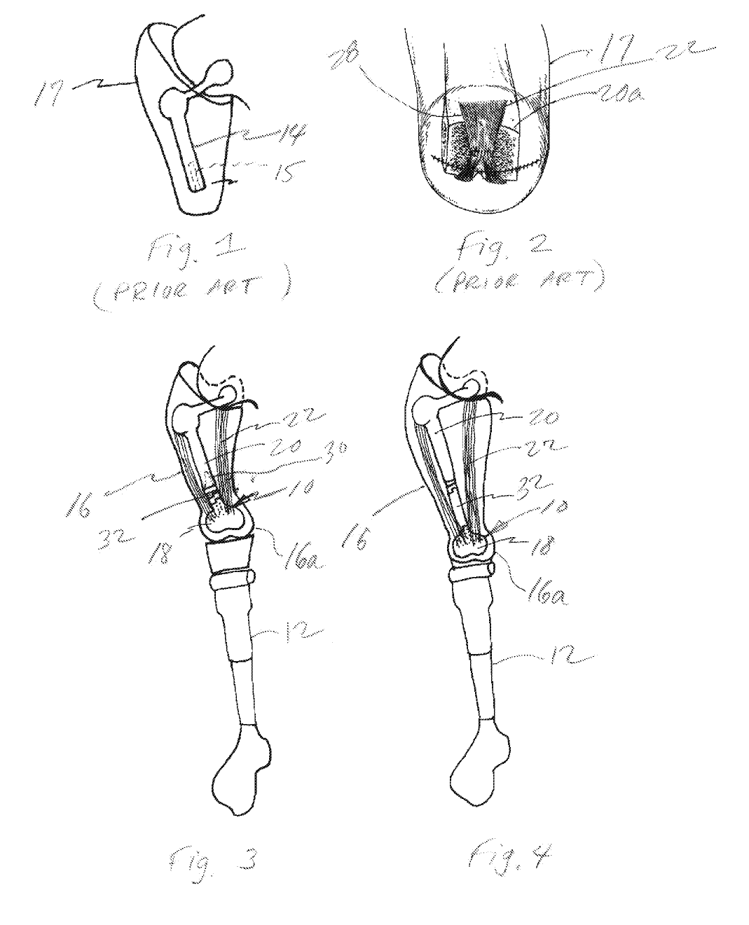

In accordance with the present invention, first referring to FIGS. 1-4, a new modular implant 10 and method for installing the same into a patient is provided for interfacing with an external prosthesis 12, such as that show in FIG. 8. The present invention provides a way to give back to cut bone trans-femural AK amputees the means to distally transfer mid-stride body weight that was lost at the time of amputation of their natural leg. While a cut end-bone 14 cannot directly bear weight, the novel surgically implanted prosthetic of the present invention create a lengthened residual limb 16 in accordance with the present invention and a condyle-like prosthetic platform, generally referred to as 18, for direct skeletal weight bearing. This represents a fundamental change and dynamic improvement in the connection between the residual limb 16 and the external prosthetic leg 12. A major secondary benefit is the opportunity to prosthetically extend the length of the residual femur 20 and provide for the reattachment of not otherwise utilized muscle 22 that become significant contributors to this longer and more powerful lever.

It should be noted that muscle reattachment following skeletal amputation can be achieved using a variety of techniques including suturing (if bone is available), meshes or other materials to provide a physical bond between the muscle and the prosthesis 10, as will be discussed below. Such materials would preferably encourage or at least not limit tissue growth into or onto the surface of the prosthesis 10. The surface preparation, materials selection, and geometry of the prosthesis surface are ways to modulate tissue ingrowth and ongrowth.

Combined with the design of a socket 24 of an external prosthetic 12, as in FIG. 8, that complements and utilizes the specifically designed part prosthetic implant 10, the result is previously levels of comfort that are not achievable in the prior art and function that provides dramatic improvement in the functional rehabilitation of above-the-knee (AK) amputees, their quality of life and consequential life-long health benefits. Significant additional benefits include vastly reduced skin breakdown, diminished gait deviation, reduced energy expenditure, increased walking speed, improved performance and endurance, and the employment of not otherwise utilized muscles.

For example, FIG. 8 shows an example of an external prosthesis 12 with a socket 24 for receipt of amputated limb 16. This external prosthesis 12 can be easily modified to accommodate the distal end of the limb 16 with the implant of the present invention. The socket 24 defined by the side walls 26 of the external prosthesis 12 can be easily altered to custom fit the free end 16a of the limb 16 of the patient with the implant 10 residing thererein.

By way of background and to illustrate the need for the present invention, knee disarticulation amputations, which are technically considered AK, retain a complete femur that in addition to having the longest possible lever to power the prosthesis, also allows efficient distal weight bearing on the broad base of the femoral condyles. All large muscles of the thigh that do not cross the knee remain attached to the distal femur and make a significant contribution to the strength and endurance of the residual limb. The residual condyles have the very important attribute of being able to directly end weight bear at the very most distal point of the residual limb. Mechanically and functionally, this is a great advantage. Further, in a properly fitting socket 24 for an amputee with a knee disarticulation, there is little or no unnatural or uncomfortable stress to the proximal soft tissue and consequently little or no soft tissue complication. In a normal anatomical knee, the femoral condyles play a critical role in load distribution to the tibia.

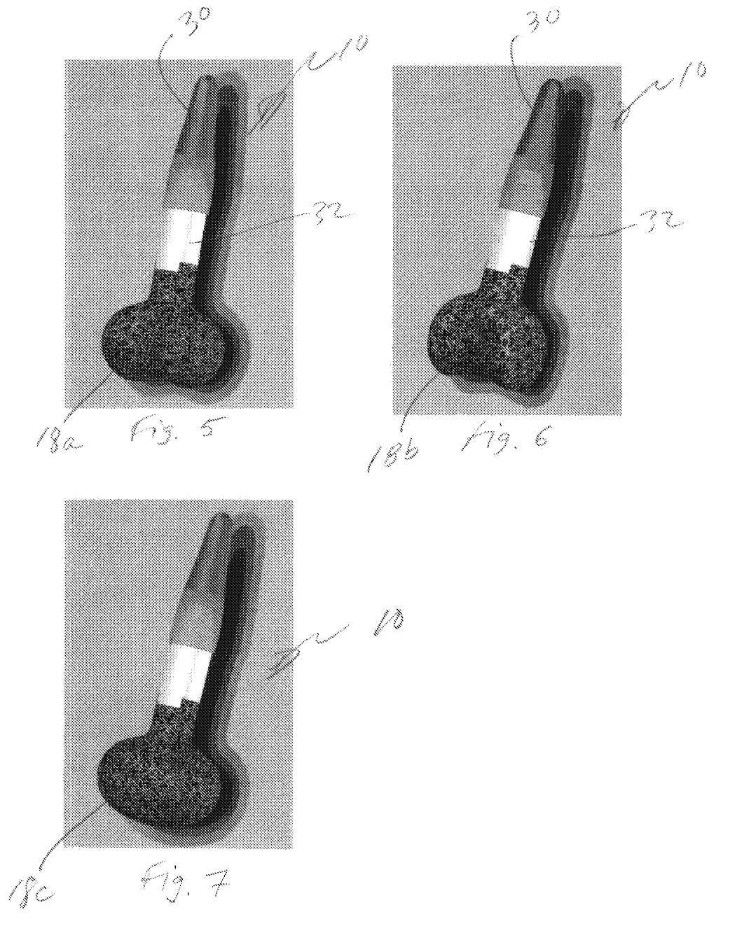

As can be seen in FIG. 3, the proximal component of the prosthetic implant 10, which may be one or more parts, is a non-cemented or cemented total joint replacement component surgically inserted into the canal 15 of the bone 14 to make an internal mechanical connection to the amputee's cut bone, such as a femur 14. The stem 30 resides in the canal 15. The condyle 18 is either attached directly to the stem 30 or to the stem 30 via an extension 32. In FIG. 3, the extension 32 is very short which is suitable in this example while extension 32 in FIG. 4 is longer to accommodate a residual limb 16 that is shorter than in FIG. 3.

Having asymmetrical knee centers makes for an awkward and unnatural looking gait in what otherwise is a functionally superior level of amputation. With the implant and system 10 of the present invention, modular component lengths overcome this principal drawback encountered in knee disarticulation amputations. In fact, the proposed prosthetic implant 10 can become a viable surgical option at the time of amputation for candidates for knee disarticulation surgery. It would result in all the advantages of true distal skeletal weight bearing while providing a residual femur length 14 that would permit equal knee center heights.

Thus, the implant 10 is modular in nature. For example, the implant of FIG. 3 includes an extension member 32 that is of a given length, as desired, to match with the length of the other leg of the patient and for proper loading, and the like. This component 32 can be produced in various lengths and configurations to allow for, among other benefits, a variety of bone diameters and a lengthening and resultant strengthening of the effective lever that powers the external prosthetic knee and lower leg 16 used by an above-the-knee amputee.

Furthermore, in the non-cemented case, various techniques are used currently to encourage bone ingrowth or ongrowth to the prosthesis using surface coatings (e.g. beads, meshes, etc.), treatments, or preparations. Again, this applies to both the fixation of the prosthesis to the cut bone and to the reattachment of muscle to the prosthesis. For example, the condyle 18 at the free end 16 is preferably made of a porous mesh material, such as that made of titanium or tantalum, to facilitate soft tissue ingrowth for permanent muscle attachment.

As seen in FIGS. 5-7, further details of the construction of the unique modular implant of the present invention is shown. In general, the implant includes a stem 30, an optional extension 32 and distal implant condyle, generally referred to as 18 where three embodiments 18a-c are shown. The distal component 18 provides a number of sizes and/or shapes of prosthetic condyles to allow for an expanded platform to enable direct distal weight bearing, surgical attachment of not otherwise utilized muscles, improved mid-stride suspension of the external leg prosthesis 12 and better residual limb 16 tissue health with less tissue breakdown as a result of improved load distribution within the prosthetic socket 24. The distal component 18 is mechanically connected to the proximal. In a further embodiment, both are assembled intraoperatively. Alternatively, component selection can be made before surgery based on imaging studies. Mechanical connection between proximal 30, 32 and distal components 18 can be accomplished using a variety of techniques, including but not limited to a Morse taper commonly used to attach, for example, the stem and ball of modern hip replacement prosthetic components.

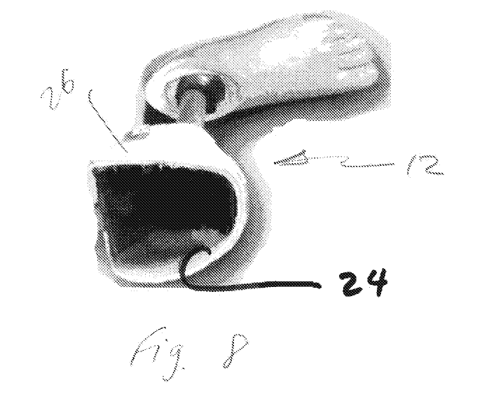

FIGS. 5-7 illustrate three different embodiments 18a-c of the present invention that include different configurations of the prosthetic condyle 18 at the free end of the implant. These different condyle configurations 18a-c are merely just examples and enable customization of the installation of the implant 10 depending on the muscle and tissue 22 location and nature thereof. For example, FIG. 5 illustrate a modular implant 10 with a prosthetic condyle 18a with two substantially spherical members while FIG. 6 illustrates a modular implant 10 with a prosthetic condyle 18b with two oblong members. FIG. 7 further shows a modular implant 10 with a prosthetic condyle 18c with a single member which may be more suitable in certain patient situations.

In accordance with the present invention, to maximize the benefits of the implanted modular implants with prosthetic condyles, a simple socket design is provided that eliminates the most problematic issues in current narrow ML design sockets. From a structural and functional standpoint, these stem from the basis of the ML socket design. Because direct vertical weight transfer is not possible at the distal cut femur 14, the moment of weight transfer is accomplished by means of an unnatural loading of soft tissue at the extremes of a roughly horizontal medial-lateral plane between the ishial ramus and the proximal femur. The unfortunate consequence of this unnatural loading of the skin and soft tissue is that they must bear the full downward force of body weight at mid-stride, during thousands of cycles each day of use. For the ML socket design to work optimally, fit is critical and must remain within a relatively small degree of tolerance. In real life, the volume of the residual limb often fluctuates during the course of a day beyond this degree of tolerance. In the short term, the result is tissue irritation and break down leading to discomfort, pain and decreased function. In the longer term, this difficulty in arriving at stable good socket fit requires frequent frustrating, expensive and time consuming return visits to a prosthetic facility. The present invention addresses these issues by providing a condyle 18 at the free end of the modular implant 10 that is large enough to distribute the load to, in turn, reduce pain for the patient.

Still referring to FIGS. 5-7, the novel modular implant 10 matches and utilizes a specific shape to maximize the functional performance possibilities of the specifically shaped prosthetic condyles 18a-c. It allows for direct weight transfer in a vertical moment at the most distal skeletal location within the prosthetic socket 24 of the artificial leg or external prosthetic 12. This will largely eliminate the previously described common experience of proximal soft tissue stress and pressure ulcers. The socket design utilizes the designed shape of the prosthetic condyle portion 18 of the modular implant 10 to minimize the additional common problem of rotation of the prosthetic socket 24 on the residual limb 16 during the weight-bearing phase of walking. It achieves improved and superior suspension during the non-weight-bearing phase of walking and will significantly reduce and potentially eliminate most or all “pistoning” of the residual limb 16 within the socket 24. This almost universal problem in narrow ML sockets also leads to soft tissue irritation, breakdown, user pain and a limiting of function.

Because the demands of unnatural skin and soft tissue loading are vastly reduced, the prosthetist may quickly reach a definitive socket fitting that will require fewer or even no adjustments over a greatly extended useable life cycle of the socket 24. By greatly lessening or eliminating the commonly inconvenient and expensive requirement for ongoing return visits to the prosthetic shop for serial adjustments and a frustratingly short useable life for each new socket 24, the system of the present invention greatly reduces the need for frequent socket replacements. Instead of the now common one to four year replacement cycle for AK sockets, the socket design (when used post-implant) for use with the present invention is targeted for five to ten years of less problematic and functionally superior socket life with a subsequent savings of many tens of thousands of health care dollars over each amputee's lifetime.

With the use of the modular implant 10 of the present invention, transfemoral AK amputees can enjoy a level of sustained comfort and function not heretofore possible for any above-the-knee amputee who does not have a knee disarticulation.

Still further, soft tissue breakdown and many common gait deviations need no longer be chronic problems in the life of an AK amputee when the modular implant 10 of the present invention is used. Instead, there is reduced energy expenditure and an increase in sustained walking speeds, endurance and overall performance. This system reduces pain, improves comfort and proprioception resulting in enhanced user satisfaction, confidence and an overall higher level of rehabilitation and health. In addition, our invention will enable recent advancements in knee and lower leg prosthetic components to produce significantly greater functional benefits for individuals with transfemoral AK amputations. This system is compatible with all existing prosthetic knee and lower prosthetic leg designs and manufactured systems available for AK amputees today. Thus, the beneficial potential described above extends to the complete range of cut end-bone amputations.

Usefulness and applicability of present invention, as applied to cut end-bone AK amputees, comes from observing the functionally superior outcomes achieved by above the knee amputations that retain the complete femur and femoral condyles (knee disarticulations). The present invention addresses a major drawback that is associated with this level of amputation. Currently, amputations that are “knee disarticulations” present a problem in that the mechanical space required below a full length femur with intact femoral condyles necessitates locating the knee center of the artificial limb lower than that of the sound side. The resultant difference in knee center heights leads to awkward gait patterns and cosmetic issues. Ideally in these cases a surgical resection is performed to remove a section of the femur while retaining the condyles so that the required space is gained to allow the placement of the prosthetic knee at an equal knee center height to that of the sound side. It may prove valuable at the time of initial amputation (or later in the cases of pre-existing knee disarticulation amputations) to cut the residual femur in such location as to allow for the fitting of our prosthetic condyles at the desired height. The advantage of a procedure performed in accordance with the present invention over an osteo re-section results in a simpler surgery with a quicker recovery than is required in the case of an osteo resection. In addition, the specifically designed shape of the modular implant, namely at the distal end at the prosthetic condyle 18 in place in the complementary socket 14 offer resistance to rotation of the artificial leg 12 on the residual limb 16. Thus, twisting of the limb 16 within the prosthesis 12 can avoided along with the pain associated therewith.

It should be understood that the present invention and system can be used not only for legs but also for arms. A similar approach and methodology to cases of upper extremity elbow and wrist disarticulations can be employed in accordance with the present invention. Here as well, it has been discovered that there are effective solutions to issues of prosthetic joint placement and overall prosthetic arm length in relation to the sound side. In that connection, the same basic concept with location specific components, both above elbow and below elbow amputees can gain a longer residual limb with not otherwise utilized muscle attachments and a distal shape that supports a simpler, lighter socket with better control and more powerful function. As can be understood, the components of the modular implant can be modified to suit the environment of an arm as compared to a leg, as shown in FIGS. 3-7.

Use of the modular implant 10 of the present invention has particular valuable use in cases of traumatic injuries and other medical conditions requiring amputation that will result in an upper extremity amputation that leaves only one to two inches of residual bone. Instead of being left with a residual limb 17, as in prior art FIGS. 1 and 2, that has neither a sufficiently long lever nor adequate muscular attachments, the limb can be reconstructed at the time of amputation using not otherwise utilized muscles and the prosthetic implant or implants. The result is an amputation, as in 16 in FIGS. 3 and 4, that instead of being “short” and functionally problematic will yield a longer and stronger skeletal/prosthetic lever with function otherwise only achievable with an amputation that retains significantly more length of that section of the limb.

There are many cases in which this means a more conventional artificial arm that is functionally more durable and significantly less expensive will exceed the function of artificial arms made of often prohibitively expensive and mechanically problematic myo-electrical components that would otherwise be desirable in cases of upper extremity amputation with short to very short residual bone length.

This system includes significant potential benefit for below the knee, also known as BK, amputees. While below the knee lower extremity amputations are able to achieve direct skeletal weight transfer utilizing the residual condyles of the tibia, if the cut bone of the distal tibia is fitted with a location specific weight bearing prosthetic implant 10, or a bridging implant is placed between the distal locations of both the tibia and the fibula cut bones providing an even larger area for direct weight transfer, this would enable the weight at mid-stride to be transferred down into the external prosthetic leg socket to the most distal location.

The transmission of any amount of weight by the cut end of the tibia and a prosthetic implant may alleviate the often painful, recurring and difficult to resolve problem that results from pressure on the popliteal nerve as it passes over the fibular head. This is pressure that comes from the current degree of limited tolerance necessary to closely capture the residual tibial condyles for total mid-stride skeletal weight transmission at this proximal location.

As in above the knee applications, the advantages of moving from proximal to distal transmission of weight include, bio-mechanically superior weight transfer for more secure and efficient locomotion, the opportunity to gain operative power from the reattachment of otherwise not utilized muscle 22 and a more “user friendly” socket fit that provides greater comfort, function and endurance with fewer return visits to the prosthetic shop for adjustments. This will result in longer socket service life with significant savings of health care dollars.

Whether transfemoral, knee disarticulation (KD), above knee (AK), below knee (BK), above elbow, elbow disarticulation, below elbow or wrist disarticulation, this apparatus and system 10 of novel implants surgically placed at the cut end-bone 20 is designed to be used with sockets 24 of external prosthetics 12 and is designed to more quickly and easily produce previously unachievable levels of fit, function, rehabilitation and user health at reduced long term cost.

In view of the foregoing, a new and novel modular prosthetic implant 10 is provided that greatly improves the interface between an amputation limb 16 and a prosthetic device 12. The modular implant 10 includes a prosthetic condyle portion 18 that uniquely provides a larger surface to receive muscle and tissue 22 for increased comfort and stability.

It would be appreciated by those skilled in the art that various changes and modifications can be made to the illustrated embodiments without departing from the spirit of the present invention. All such modifications and changes are intended to be covered by the appended claims.

What is claimed is:

1. A modular prosthetic implant for a limb that interfaces with an external prosthesis, comprising:
a stem; and
a prosthetic condyle connected to the stem; the condyle being capable of receiving tissue thereon.

2. The modular prosthetic implant of claim 1, further comprising:
an extension member disposed between the stem and the prosthetic condyle.

3. The modular prosthetic implant of claim 2, wherein the extension is adjustable in length.

4. The modular prosthetic implant of claim 1, wherein the stem and prosthetic condyle are integrally formed.

5. The modular prosthetic implant of claim 1, wherein the prosthetic condyle is manufactured of metal.

6. The modular prosthetic implant of claim 3, wherein the metal is porous.

7. The modular prosthetic implant of claim 1, wherein stem is receivable into the canal of a bone.

8. The modular prosthetic implant of claim 1, wherein the prosthetic condyle simulates the anatomy of an actual condyle.

9. The modular prosthetic implant of claim 1, wherein the prosthetic condyle distributes the load placed thereon.

10. The method of installing a modular prosthetic implant, comprising the steps of:
providing a stem;
providing a prosthetic condyle connected to the stem;
inserting the stem into a canal of a bone;
attaching muscle to the prosthetic condyle;
closing the skin over the muscle and prosthetic condyle;
mating the prosthetic condyle, with skin and muscle thereover, with a prosthesis.

11. The method of claim 10, further comprising the step of:
providing an extension member between the stem and the prosthetic implant;

12. The method of claim 11, wherein the extension is adjustable in length.

13. The method of claim 10, wherein the prosthetic condyle is manufactured of metal.

14. The method of claims 13, wherein the metal is porous.

15. The method of claim 10, wherein the prosthetic condyle simulates the anatomy of an actual condyle.

16. The method of claim 10, wherein the prosthetic condyle distributes the load placed thereon.
